(19) United States
(12) Patent Application Publication
(10) Pub. No.: US 2018/0039328 A1
(43) Pub. Date: Feb. 8, 2018

(54) BIOMEDICAL SYSTEM VARIABLY CONFIGURED BASED ON ESTIMATION OF INFORMATION CONTENT OF INPUT SIGNALS

(71) Applicant: The General Hospital Corporation, Boston, MA (US)

(72) Inventors: Wasim Qamar Malik, Boston, MA (US); Leigh Robert Hochberg, Boston, MA (US); Emery Neal Brown, Boston, MA (US)

(21) Appl. No.: 15/549,358

(22) PCT Filed: Apr. 21, 2015
(86) PCT No.: PCT/US15/26754

§ 371 Date: Aug. 7, 2017

Related U.S. Application Data
(60) Provisional application No. 61/982,121, filed on Apr. 21, 2014.
(60) Provisional application No. 62/065,940, filed on Oct. 20, 2014.

(51) Int. Cl. G06F 3/01; A61B 5/0484; A61B 5/00

(52) U.S. Cl. CPC G06F 3/015; A61B 5/4064; A61B 5/7203; A61B 5/0484; A61B 5/7475; A61B 5/0006

(57) ABSTRACT

Computationally efficient procedure for estimating modulation depth from multivariate data. Neural interface system utilizing such procedure to rank neural signals and select optimal channel subset for inclusion in the neural decoding algorithm. The proposed system offers several orders of magnitude lower complexity and data-processing time but virtually identical decoding performance compared to greedy search and other selection schemes and is applicable to wide variety of problems involving multisensor signal modeling and estimation in biomedical engineering systems. The use of the system to the modulation depth of human motor cortical function shows that single-unit signals are characterized by the generalized Pareto distribution.

### CROSS-REFERENCE TO RELATED APPLICATIONS

The present international application claims priority from U.S. Provisional Patent Applications Nos. 61/982,121 filed on Apr. 21, 2014, and 62/065,940 filed on Oct. 20, 2014. The disclosure of each of the above-referenced patent applications is incorporated herein by reference.

### TECHNICAL FIELD

The present invention relates in general to biomedical systems that include interfaces with a biological tissue and, in particular, to neural systems in which neural channels are selected based on the information content of signals acquired through the channels from the tissue.

### BACKGROUND

Rapid developments in technology of biomedical systems and, in particular, in neural interface technology are making it possible to record increasingly large signal sets of neural activity. Various factors such as asymmetrical information distribution and across-channel redundancy may, however, limit the benefit of high-dimensional signal sets representing a space of large biomedical parameters, and the increased computational complexity may not yield corresponding improvement in system performance.

Such is the case, for example, with a popular approach used to reduce the dimensionality of signals acquired by neural interfaces, the so-called “variable selection” methodology. According to this methodology, the subsets of signals acquired with a neural interface are identified and ranked based on signal-to-noise ratio (SNR), or information content of the channels, or another characteristic that is important from the viewpoint of estimation and detection of the signal. Projection techniques used for variable selection of neural information channels—such as principal component analysis (PCA), or independent component analysis (ICA), or factor analysis—involve a transformation to an optimal bases and produce abstract features that are inevitably disconnected from their underlying neurophysiological meaning, making intuitive interpretation of the results of processing of neural information (the very goal of an interface system) difficult. Furthermore, since input channels of an interface system are used to compute the projections by combinations (in one example—linear combinations), data from each and every one of the channel must be recorded and preprocessed. Moreover, since projection techniques such as the PCA do not take the behavioral correlate into account, the largest principal components or channels identified by such techniques are recognized to be often simply unrelated to the task conditions.

It is appreciated, therefore, that the preferred method for selecting the optimal neural information channels from the plethora of channels available for collecting the neurological information should be devoid of such transformations, and should operate instead in the original space of physical channels, providing data that can be clearly and intuitively correlated with neurophysiological processes taking place.

### SUMMARY

Embodiments of the invention provide a biomedical system for transforming activity signals acquired from a biological tissue. Such system includes an input interface unit, preprocessing electronic circuitry, an estimator unit, and a channel subset selector. The input interface unit is configured to simultaneously receive a multiplicity of raw signals from the array of signal channels; a preprocessing electronic circuitry transforms said raw signals to refined signals (by at least amplifying the raw signals, filtering said raw signals to reduce noise, and detecting a predetermined feature in the raw signals). The estimator unit is structured to determine information content of each of the signal channels from the array. The channel subset selector in operable communication with the estimator unit received values representing the information content from the estimator unit, based on these values that have been ordered, generates a marker representing a limit on a number of the signal channels to be used in the system. In one embodiment, the activity signals include neural activity signals and raw signals include electrical signals. In a specific embodiment, information content includes a modulation depth of each of the signal channels and, in addition, the channel subset selector is configured to receive, in operation, values of modulation depths of each of the signal channels from the array to generate the marker when a first sum of these values exceeds a predefined threshold. Alternatively or in addition, the channel selector identifies numbers of channels by adding successive values of the ordered (in a descending order) modulation depth values until the cumulative sum exceeds a predetermined threshold. In a case when the estimator unit contains electronic circuitry programmed to determine a modulation depth of a channel, such modulation depth is defined as a ratio of i) a response of said chosen signal channel to a behavioral variable to ii) a spontaneous activity of said chosen signal channel and noise. The behavioral variable includes a pre-determined input provided to the biological tissue and, depending on the implementation, such input may contain any of an optical stimulus, an auditory stimulus, a tactile stimulus, a gustatory stimulus, and an olfactory stimulus. In a related embodiment, the biomedical system further includes a decoder unit, in electrical communication with the channel subset selector and the preprocessing electronic circuitry, and an endeffector operably connected to the decoder unit. The decoder unit is configured to acquire first refined signals corresponding only to signal channels from a subset of the array identified by the marker, and to decode said first refined signals in a fashion that is suitable to generate a required response from the endeffector subjected to decoded refined signals. In a specific case when the endeffector includes a tissue (and not a purely prosthetic device), the decoder unit decodes the chosen refined signals to generate such a physiological response from the tissue of the endeffector that mimics a pre-determined input that has been provided to the biological tissue, and based on which the information content of a channel from the array is determined by the estimator unit.

Embodiments further provide a method for operating a biomedical system, which method includes a) reducing a dimensionality of an ensemble of multiple signal channels (through which activity signals are acquired from a biological tissue that has been subjected to an input) by selecting a signal channel subset without employing a projection-based technique; and b) decoding a subset of the activity signals acquired through the signal channel subset to obtain a first output that differs in a predetermined fashion from to a second output. In one embodiment, the first output represents a decoding correlation obtained as a result of decoding said subset of said activity signals and the second output includes a decoding correlation obtained as a result of decoding all activity signals. The first output, depending on the particular embodiment, includes at least one of optical, electrical, magnetic, mechanical, and acoustic actions and/or signals. In a specific case of such embodiment, a difference between the first and second outputs does not exceed 0.1. The second output is defined as a result of decoding of activity signals acquired through the entire ensemble of multiple signal channels.

In a specific case, the first output includes a decoding correlation obtained as a result of decoding said subset of the activity signals, and the second output includes a decoding correlation obtained as a result of decoding all activity signals. In one embodiment, the step of reducing dimensionality includes performing variable ranking of the multiple signal channels in an original signal space in which a physiological meaning of the multiple signal channels is defined. Alternatively or in addition, the step of reducing dimensionality may include determining information content for each of the multiple signal channels and, in a specific implementation, such step includes a determination of modulation depths of the multiple signal channels and ranking these multiple signal channels based on determined modulation depths, transforming raw signals acquired through the ensemble to refined signals by at least amplifying said raw signals, filtering said raw signals to reduce noise contained therein, and detecting a predetermined feature in said raw signals; and decoding only those refined signals that correspond to multiple signal channels identified by said numbers, said decoding effectuated in a fashion that is suitable to generate a required response from an endeffector subjected to decoded refined signals. In one embodiment, decoding is effectuated in a fashion suitable to generate a required response from the endeffector. Alternatively or in addition, the method includes a step of subjecting the biological tissue to a pre-determined input that includes at least one of an optical stimulus, an auditory stimulus, a tactile stimulus, a gustatory stimulus, and an olfactory stimulus. When the endeffector contains tissue, the decoding may be specifically effectuated to generate a physiological response from such tissue, which physiological response mimics the pre-determined input to the biological tissue.

### BRIEF DESCRIPTION OF THE DRAWINGS

The disclosure presented in the Detailed Description section of this application will be better understood in conjunction with the following generally not-to-scale Drawings, of which:

FIGS. 1A and 1B provide a schematic representation of a variable-selection scheme and behavioral task design for neural recordings. FIG. 1A: The decoder in a neural interface with optimal variable subset selection based on modulation depth ranking. FIG. 1B: Open-loop center-out-back task for recording neural activity under motor imagery, with four peripheral targets and a computer-controlled cursor. Bounding rectangle represents the computer screen and scale bar is in units of visual angle.

FIGS. 2A and 2B illustrate modulation depth of human motor cortical single-unit spike-rates. FIG. 2A: Distribution of modulation depth (radial length) and preferred direction (angle) of 39 individual channels (single-units) recorded in one research session. FIG. 2B: Cumulative modulation depth as a function of optimal subset size. Different dashed lines represent numbers of channels required to achieve at least 50%, 90% and 95% of total modulation depth, respectively.

FIGS. 3A and 3B illustrate decoding with the use of the optimal channel subset. FIG. 3A: Open-loop center-out-back task with rightward peripheral target (gray circle). Straight horizontal line: computer cursor trajectory from home position outward to target and back; Curved line: cursor trajectory estimated by decoding neural activity of the m best channels ranked by modulation depth; black rectangle: computer screen location and dimensions; scale bar in units of visual angle. FIG. 3B: Decoding of the system's hidden state (imagined cursor velocity) from neural activity using m best channels. The trials, each of 12 s duration, start with target onset at time “zero”. Arrow: direction of selected target relative to home position.

FIGS. 4A, 4B, 4C provide illustration to the effect of neural-channel modulation depth on decoding performance. FIG. 4A: Relation of modulation depth to open-loop decoding correlation of true computer cursor velocity with velocity estimate obtained by decoding each neural channel individually. Shaded region: 95% CI of chance-level decoding correlation. FIG. 4B: Improvement in decoding correlation with increasing channel subset size chosen using the specified scheme. FIG. 4C: Normalized Bayesian information criterion (BIC) as a function of channel subset size chosen by modulation depth. Red circle: optimal subset size with lowest BIC.

FIGS. 5A, 5B, and 5C and 5B illustrate the effect of excluding some of the highest modulated channels from consideration on decoding performance. FIG. 5A: Decrease in total modulation depth; FIG. 5B: open-loop decoding correlation between true and estimated cursor velocity (bottom) when the specified number of the best channels are removed from the neural decoder. Shaded region: 95% CI of chance-level decoding correlation. FIG. 5C: Open-loop center-out-back cursor trajectories decoded from m channels with the lowest modulation depth. Straight lines 530: computer cursor trajectory; Curved lines 534: imagined velocity decoded from neural channel subset; black rectangle: computer screen.

FIGS. 6A, 6B, 6C provide statistical characterization of the single-unit modulation depth. FIG. 6A: Distribution of modulation depth of various channels in each of 5 research sessions. Line 610: median; box: interquartile range [q₁,q₃]; whiskers: extreme points that are not outliers and lie within the range q₃±1.5 (q₃−q₁); circles: outliers. FIG. 6B: A histogram of modulation depth data from 5 research sessions, along with the scaled best-fit generalized Pareto probability density function. FIG. 6C: Cumulative distribution function of modulation depth data and best-fit generalized Pareto distribution.

FIG. 7 is a flow-chart of an embodiment of the method of the invention.

### DETAILED DESCRIPTION

The present invention pertains to a process for selection of neural signals with the purpose of eliminating low-information channels to improve the computational efficiency and generalization capability of a biomedical system and the corresponding biomedical interface system. The problem of identifying the most preferred signal channels (chosen, in one non-limiting example, from the available plethora of channels transmitting neural activity to a neural interface system) and finding the optimal tradeoff between model complexity and performance of the neural interface system is solved by ranking and selecting the channels according to the information content of signals transmitted through these channels (in one specific example—the depth of modulation of such signals). Embodiments of the invention offer a biomedical system characterized by several orders of magnitude lower complexity but virtually identical decoding performance as compared to the systems utilizing the now popular greedy search or stepwise regression, which includes forward selection (C. Vargas-Irwin et al., J. Neurosci., vol 30, no. 29, pp. 9659-9669, July 2010; J. Zhuang et al., IEEE Trans. Biomed. Eng., vol. 57, no. 7, pp. 1774-1784, July 2010), backward elimination (J. Wessberg et al., Nature, vol. 408, no. 6810, pp. 361-365, November 2000), or selective neuron dropping (J. C. Sanchez et al., IEEE Trans. Biomed. Eng., vol. 51, no. 6, pp. 943-953, June 2004). As a result of removal from the consideration by the neural interface system the channels characterized by low modulation depth, the embodiments of the invention increased SNR.

In one specific example, which is used to illustrate the principle of the invention, the biomedical system includes a neural interface. Neural interfaces, also referred to as brain-machine interfaces (BMI) or brain-computer interfaces (BCI), offer the promise of motor function restoration and neurorehabilitation in individuals with limb loss or tetraplegia due to stroke, spinal cord injury, limb amputation, ALS or other motor disorders. A neural interface system infers motor-intent from neural signals, which may include a single-unit actions potential (or spike), multiunit activity (MUA), a local field potential (LFP), an electrocorticogram (ECoG), or an electroencephalogram (EEG), for example. The interpreted by the interface movement intention is then converted into action by means of restorative technology (such as functional electrical stimulation) or assistive technology such as a computer cursor, robotic arm, wheelchair controller, or exoskeleton). Recent clinical studies (such as L. R. Hochberg et al., Nature, vol. 442, no. 7099, pp. 164-171, July 2006, for example) have also demonstrated the potential of chronically implanted intracortical neural interface systems for clinical rehabilitation of tetraplegia.

The term channel may refer to a single-unit or an electrode on a microelectrode array, for example. Recent advances in neural interface technology made is possible to simultaneously record neural activity from hundreds of channels, which has a downside because the features, or variables, derived from the recorded neural signals may form a vary large space of data. For example, each variable representing an LFP may include power data received from one of several frequency bands in the signal recorded at each electrode. This problem of an often-overwhelming amount of data is further compounded because typical neural decoding algorithms (such as a cascade Weiner filter or a point-process filter, for example) use multiple parameters per input variable. A large parameter space can severely affect the generalizability of the model due to overfitting, while model calibration and real-time decoding can simply become computationally prohibitive and impracticable. The need for a computationally efficient variable selection scheme has been recognized to become even more pronounced when a neural interface employs frequent filter recalibration to combat neural signal nonstationarity. Several methodologies have been developed to date to address the reduction of the signal dimensionality.

One of such methodologies, referred to as variable selection (or feature selection or signal subset selection) involves ranking and identifying the optimal subset of neural signals on the basis of their characteristic(s) important from an estimation and detection viewpoint (I. Guyon et al., Am. J. Mach. Learn. Res., vol. 3, pp. 1157-1182, 2003). Variable selection modality that utilizes the popular linear transformation to an optimal basis suffers from disconnect between the resulting abstract features from underlying neurophysiological meaning, and the need to record and preprocess data acquired along each and every channel (resulting in a very complex and exhaustive search method). An example of a variable selection algorithm that does not involve linear transformation and operate, instead, in the original space of data representing actual physical channels is provided by a so-called greedy search or stepwise regression.

### Apparatus.

The idea of the present invention stems from the realization that a highly-efficient variable selection scheme results from estimation of information content of a channel. (In a specific case when the modulation depth of the channel is used as metric of its information content, such modulation depth is defined as the ratio of its task-dependent response of the channel to its spontaneous activity and noise; for example, within Gaussian linear dynamical framework). As such, the information content of the channel is employed not only as a measure of each variable's relative importance to the process of decoding in a neural interface system, but also to identify the optimal variable subset.

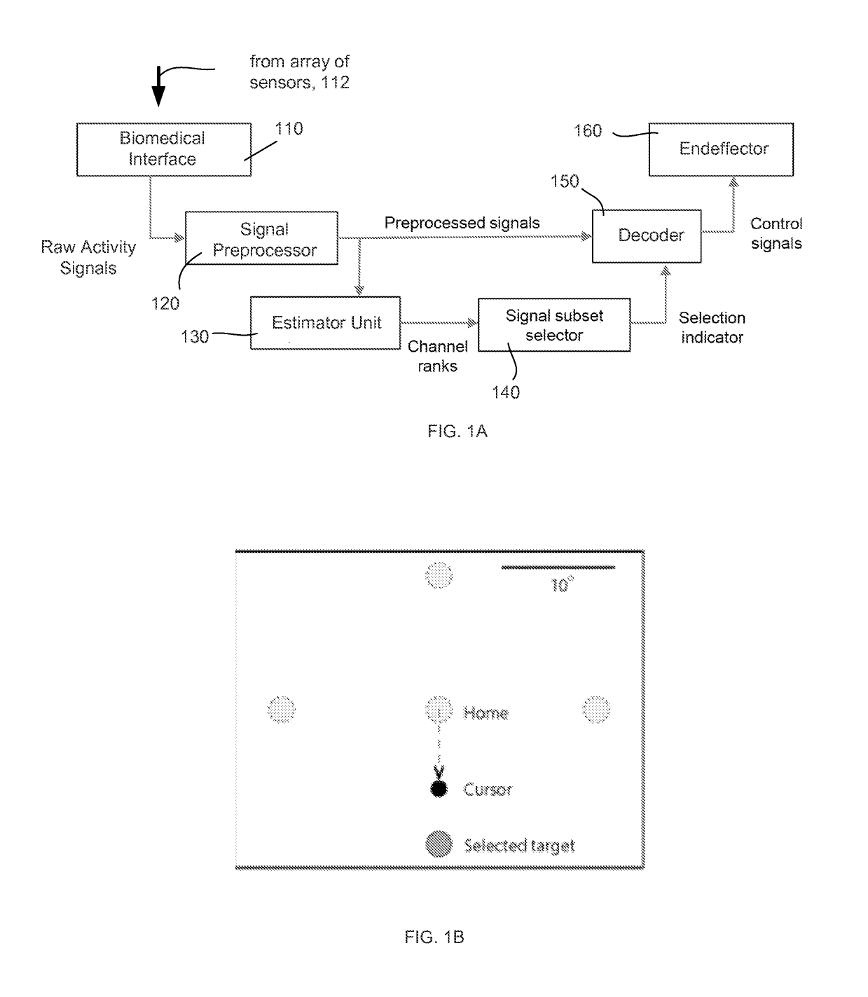

FIG. 1A provides a generalized schematic diagram of an embodiment 100 of the biomedical system of the invention employing electronic circuitry wired to variably select signal channels according to an idea of the invention. The system 100 includes a biomedical interface (in a specific example—a neurophysiological interface unit) 110 containing electronic circuitry configured to acquire activity signals (in a nonOlimiting example—neural signals) through the predetermined large number of channels 112 (such as an array of microelectrodes, in one example) and produce output signals (for example, electrical signals) representing raw activity. The unit 110 is operably cooperated with a signal preprocessor 120, which is programmed at least to amplify raw signals, filter the signals to remove noise, and perform detection of predetermined features (such as spikes in the signals, in one embodiment). (In an example of recording the neural activity in the brain, the array of neural channels and the corresponding data-acquisition system may be represented by a Cerebus™ system from Blackrock Microsystems. While the following disclosure provide examples of brain signals and a corresponding brain-machine interface systems, it is appreciated that the uses of the disclosed concepts with respect to other types of biological activity/signals/channels include magnetic channels, optical channels, auditory channels, tactile and/or gustatory and/or olfactory channels. For example, the magnetic channel implementation maybe realized in a situation when a magnetic encephalogram is being recorded; the purely electrical channels would be appropriate when measuring the EEG outside the head of the subject; the recordation of neuronal activity optically may be used in a situation employing fluorescent microscopy/two-photon microscopy, for example). The estimator 130 receives the preprocessed data output from the preprocessor 120 and transforms this output to not only determine the information content of each of the neural channels but also to order the neural channels according to the corresponding modulation depths (whether in ascending or descending order). Based on such order, the electronic circuitry of the signal subset selector 140 defines a channel filter the operational passband of which includes only a predetermined number of the ordered channels having the information content value higher than a threshold value. The so-defined filtering function is further used by the decoder 150 as a mask, according to which the only preprocessed signals (that are received by the decoder 150 from the preprocessor 120 and that are being further decoded as signals used to control the endeffector 160) are those contained within the passband of the operational filter of the selector 140. The endeffector 160 includes, in one implementation, a prosthetic device, in a related embodiment, the endeffector 160 includes a biological tissue.

### Embodiments

In the following example, the biomedical system includes the neural interface system model which, according to embodiments of the invention, was used to perform a two-dimensional motor imagery task of a prosthetic device (such as a prosthetic arm, in one example), while the intended movement kinematics were estimated from the neural activity during the performance of such two-dimensional task with the used of a state-space model and estimation of information content of a channel (the metric for which, in this example, was a modulation depth of the channel). Mathematical modeling and methods used for data-processing are described below in Section Methodology.

This clinical study was conducted under an Investigational Device Exemption and Massachusetts General Hospital IRB approval. Neural data were recorded with the neural interface 110 from a subject who had tetraplegia and anarthria (resulting from a brainstem stroke that occurred nine years prior to her enrollment in the trial) with the use of a 96-channel microelectode array disposed in the motor cortex of the subject, in the area of arm representation. The data were recorded in separate research sessions conducted on five consecutive days, from Day (n) to Day (n+4). The task that the subject was performing was formulated as a “center, out, and back” motor task, in which the subject observed and imagined controlling a computer cursor moving on a 2-D screen towards four pre-determined radial targets, schematically shown in FIG. 1B. In doing so, a single pseudo-randomly selected target was highlighted, and the computer-controlled cursor moved toward the target and then back to the home position with a Gaussian velocity profile. This constituted one trial, and this process was repeated 36 times within 4 blocks for a total of about 6 min in each session. This motor imagery task was performed under the open-loop condition, when the participant was not given control over cursor movement during the epoch. The cursor position and wideband neural activity were recorded throughout the task duration. The neural data were band-pass filtered (between about 500 Hz and about 5 kHz), and spike-sorted to identify putative single-units (in one case—neurons). For decoding analysis, these data were used to calibrate a steady-state Kalman filter (W. Q. Malik et al., IEEE Trans. Neural Sys. Rehab. Eng., vol. 19, no. 1, pp. 25-34, February 2011) and estimate the imagined movement velocity of the cursor using leave-one-out cross-validation.

The intended velocity of movement of the prosthetic device was represented generally by the n-dimensional latent variable, x(t), which in the case of a two-dimensional motor imagery task was chosen to be a two-dimensional vector, the dimensionalities of which included the horizontal and vertical components of movement of the computer cursor at discrete times t. The vector time-series of observations (for example, single-unit binned spike-rates) at time t are denoted with m-dimensional vector y(t), where m is the number of channels (in one case—single units). In practice, the single-unit spikes (action potentials) were collected through non-overlapping time-bins or time-windows of duration Δt=50 ms to obtain binned spike-rates, y(t), which were centered at zero by mean subtraction. This system exhibited short-term stability and convergence (according to Malik et al., 2011), the state-space system matrices {Φ,H,Q_{d},R_{d}} were assumed to be time-invariant. Using the Neural Spike Train Analysis Toolbox (nSTAT) (I. Cajigas et al., J. Neurosci Methods, vol. 211, no. 2, pp. 245-264, November 2012), the maximum-likelihood estimates of the system parameters were obtained as part of filter calibration with training data. The expectation-maximization (EM) algorithm was used for state-space parameter estimation, which was initialized with ordinary least squares (OLS) parameter estimates obtained using the procedure described in (Malik, 2011) As a result of OLS-based parameter initialization, the EM algorithm consistently converged in under 5 iterations. The steady-state SNR matrix S, and the modulation depth sᵢ=[S̅]_{i,j} for channels i=1, . . . , m, were estimated with the use of Eq. (18) below. To prevent numerical errors, S was constrained to be no smaller than zero.

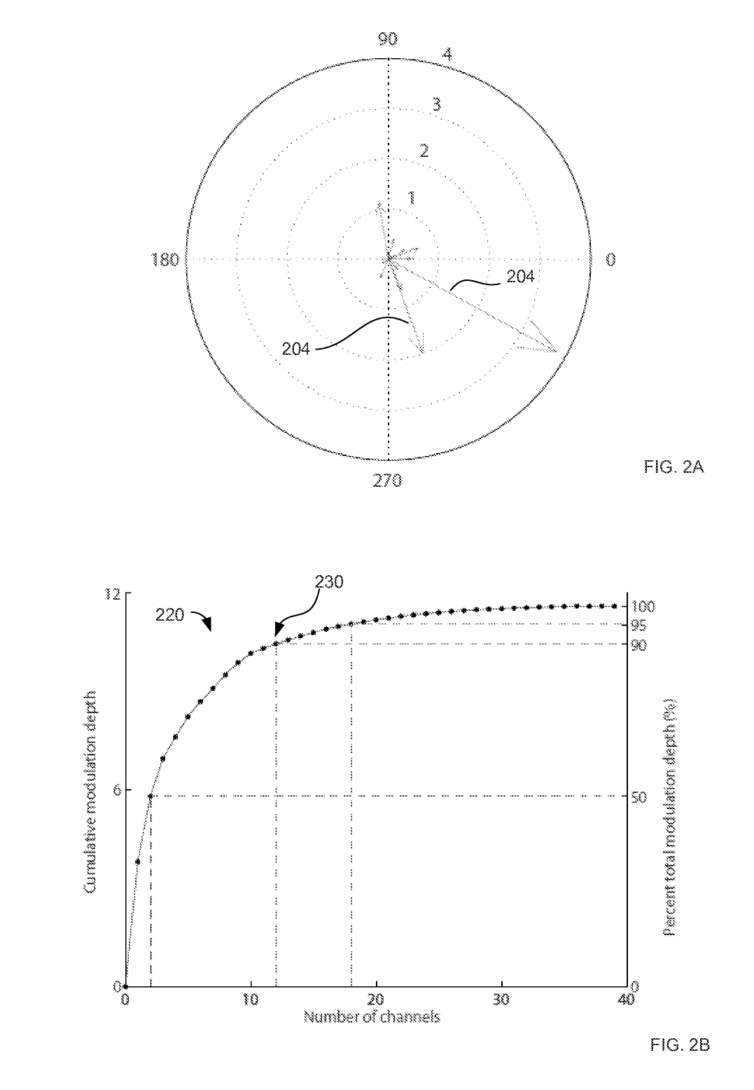

The modulation depth characteristics of an ensemble of single-units were investigated as discussed below. Unless otherwise specified, the discussed representative results are from the session conducted on day (n+4). The neural data from the session included the spiking activity of m=39 putative single-units isolated by spike-sorting. As shown in FIG. 2A, the magnitude of the modulation depth, determined for each of the 39 channels, was found to be highly asymmetrical across the channels. The modulation depth of the best (that is, the most strongly modulated) channel (s=3.8) was nearly twice as high as that of the next best channel (s=2.0). The skewness of the modulation depth distribution was estimated to be 3.5 (unbiased estimate, bootstrap).

The preferred direction of the i^{th} channel in the 2-D Cartesian space was defined as Q̅ᵢ=tan⁻¹(h_{i,2}, h_{i,1}). The preferred directions of the individual channels appeared to be distributed approximately uniformly in the (0° to 360°) range when the modulation depths (corresponding to the radial lengths of the vectors 204 in FIG. 2A) were disregarded. Modulation depth, however, is instrumental in indicating that only a small number of channels are principally relevant for decoding, and the preferred-direction spread of those high modulation depth channels is of key importance. The preferred directions of the three best channels were estimated as 330°, 290°, and 100°, respectively. Although the preferred directions of the most strongly modulated channels did not form an orthogonal basis in 2-D Cartesian space, these preferred directions were not clustered together. The obtained result suggested that for this two-dimensional motor imagery task a small number of channels—with large modulation depths and substantial spread in preferred direction—is adequate for representing movement kinematics in the 2-D space.

In addition, the increase in total modulation depth with increasing ensemble size was determined by analyzing the cumulative modulation depth curve 220 shown in FIG. 2B. The curve 220 represents a result of adding successive values of modulation depth sorted in the descending order. As the modulation depth distribution is highly nonuniform, the increase in ensemble size follows the law of diminishing returns. It was found that 2, 12 and 18 channels yielded 50%, 90% and 95%, respectively, of the total modulation depth obtained with all m=39 channels.

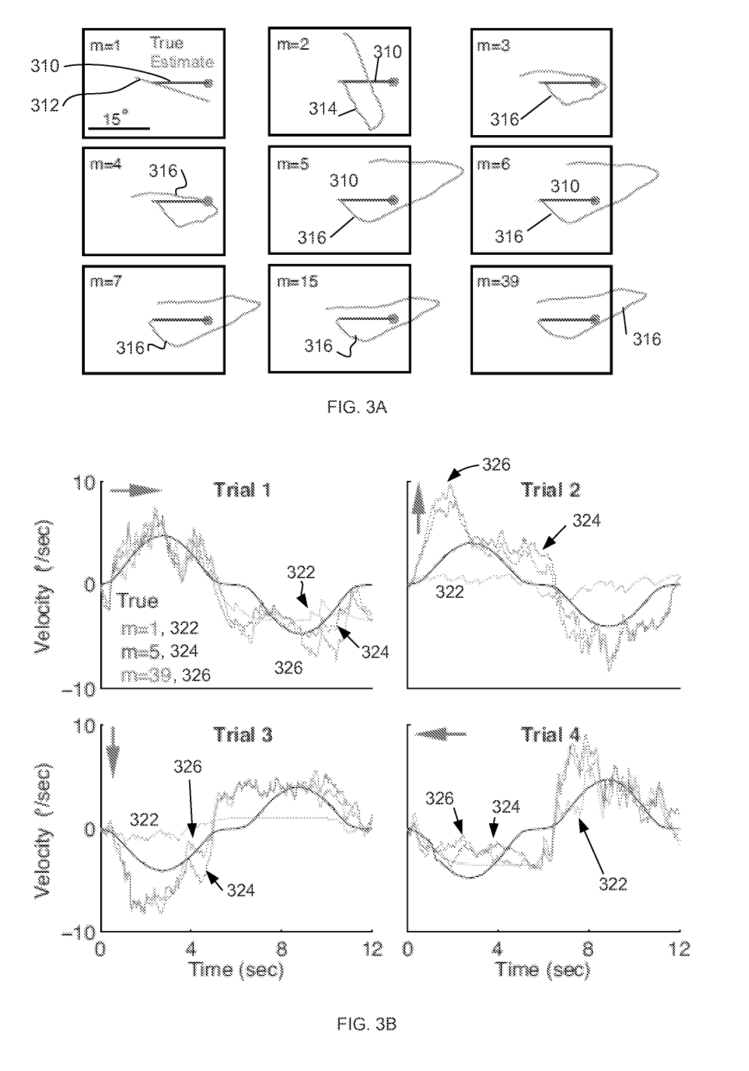

In order to address the problem of subset selection that is optimal for decoding in a neural interface system 100, the effect of ensemble size on decoding performance was studied. The results are shown in FIG. 3A for different number of channels (in comparison with the line 310 representing the computer-cursor trajectory from the home-position outward to target and back). When only the most strongly modulated channel was used for decoding (m=1), the decoded trajectory 312 was restricted to a one-dimensional axis. This 150°-330° axis was defined by the preferred direction of the corresponding channel. When the two best channels (m=2) were used for decoding, the trajectory estimate 314 spanned both spatial dimensions. The trajectory estimates 316 improved with more channels (m=3, 4, 5, 6, 7, 15, and 39) but the improvement associated with an increase in selected subset size rapidly saturated. Quite unexpectedly, the empirical results (of FIG. 3A) established that the trajectory estimate obtained by decoding open-loop motor imagery data according to an embodiment utilizing the modulation-depth approach of the invention, the neural activity remarkably follows and reflects the sequence of phases of the actual motor task (that is, the outward movement, the reversal of direction, and the inward movement) along the correct axis even in the absence of feedback control.

FIG. 3B provides plots comparing the estimates of movement velocity obtained with a single (most strongly modulated) channel, 5 of the most strongly modulated channels, and all 39 channels. In trials where the cursor movement axis was closer to the preferred direction of the strongest channel, the single-channel decoder provided reasonable velocity estimates (Trials 1 and 4 in FIG. 3B). Such result is explained by the fact that the horizontal projection of the encoding vector of that channel (shown in FIG. 2A) provided significant movement information (and was, accordingly, the dominant component of the encoding vector). At the same time, the performance of the single-channel decoder was unsatisfactory in trials with movement along the vertical axis (Trials 2 and 3 of FIG. 3B), which was orthogonal to the dominant component of the encoding vector for that channel, as expected. However, with m=5 channels, the decoded velocity was already qualitatively indistinguishable from that obtained with the entire ensemble.

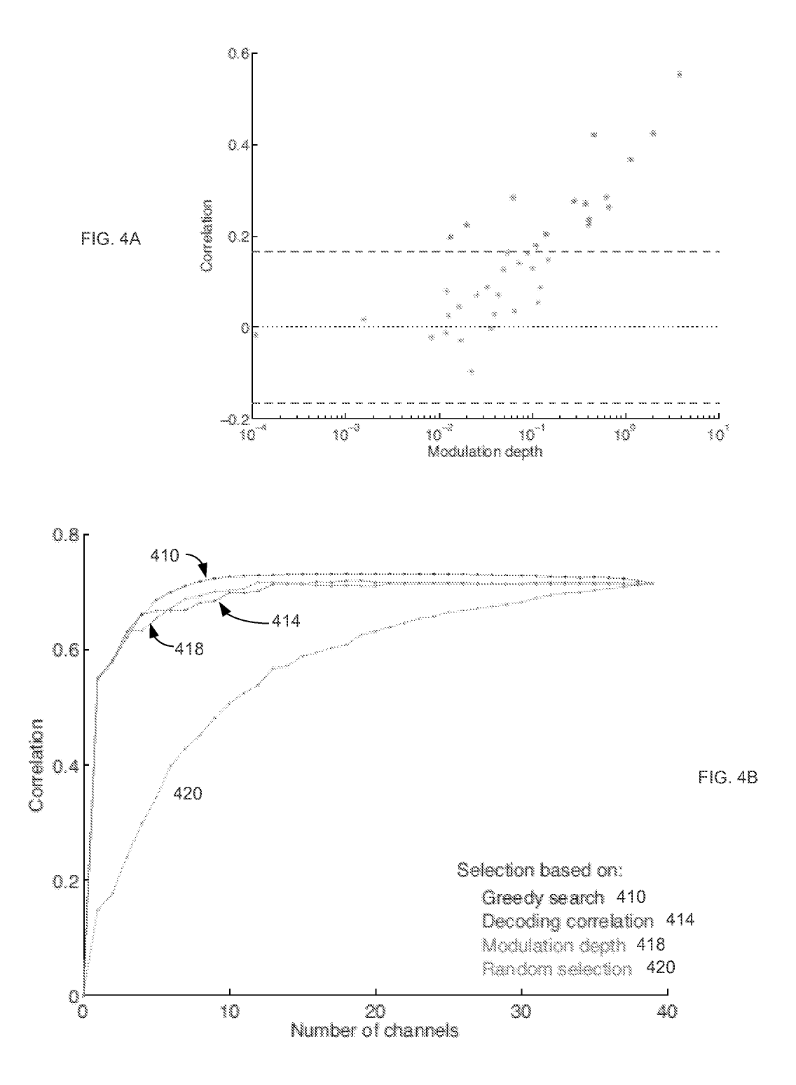

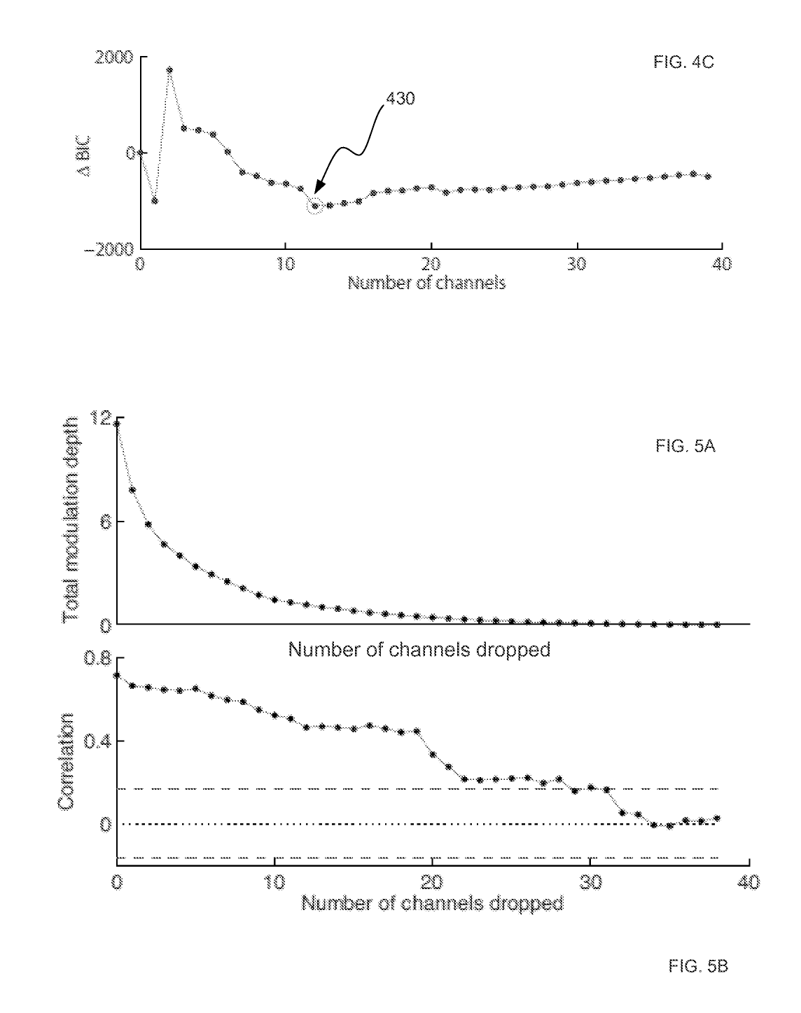

The relation between the modulation depth and decoding performance of a given channel was characterized quantitatively. To this end, the vector-field correlation was analyzed between “true” (computer-controlled) and decoded 2-D velocity of the cursor. In doing so, each channel was used individually to calibrate the filter and decode under cross-validation. The computed correlation is shown in FIG. 4A. To obtain chance-level correlation, the surrogate data was generated by bootstrapped phase randomization. The most strongly modulated channels provided significantly larger decoding correlation than weakly modulated channels. The single-channel correlation appeared to follow an exponential relationship with modulation depth for a few channels in the high-modulation depth regime, but did not have a significant relationship in the low-modulation depth regime.

To study the implications of channel ranks on decoding performance, we compared the true and decoded state correlation obtained with a subset of channels that have been selected on the basis of i) modulation depth and ii) individual channel correlation (FIG. 4B). As illustrated in Table I, the strongest modulated channel alone provided a correlation of over 0.5, and the correlation exceeded 90% of its limiting value of 0.7 with the use of m=5 highest modulation depth channels. The term decoding correlation refers to the correlation coefficient between the estimated output and a reference (or “ground truth”) value of the output.

TABLE 1
Comparison of execution time and decoding correlation
(using selected 5 of the available 39 channels) of various
selection schemes on a standard personal computer.

| Selection Scheme | Execution Time, mean ± s.e. (sec) | Correlation |
|---|---|---|
| Greedy search | 1.8 × 10³ ± 7.2 | 0.69 |
| Decoding correlation | 1.2 × 10² ± 3.8 × 10⁻¹ | 0.67 |
| Modulation depth | 4.7 × 10⁻⁴ ± 1.4 × 10⁻⁵ | 0.65 |
| Random selection | 5.8 × 10⁻⁶ ± 9.4 × 10⁻⁸ | 0.35 |

In reference to FIG. 4B, additionally evaluated was the decoding correlation achieved with greedy selection, 410, and random selection, 420, methodologies, in order to obtain upper and lower bounds, respectively, on the performance of the embodiment of the present invention. The decoding performance of channel subsets chosen by correlation 414 and modulation depth ranking 418 (i.e., according to an idea of the invention) performed almost identically. The decoding performance of these two schemes was nearly as good as that of greedy selection 410, but substantially better than random selection 420. Notably, the difference between the results achieved with random selection 420 and the embodiment of the invention 418 reduced as the selected subset size increased. (It is understood than when information acquired by a lot of or all of the channels is used, the effect of intelligently selecting channels is not present).

Further, and in reference to FIG. 4C, the optimal number of channels to be included in the system—in terms of the tradeoff between decoding accuracy and model complexity—was investigated with the use of a fitness metric, which in this case included the Bayesian information criterion (BIC). According to Eq. (2) discussed in Methodology, the addition of each extra channel contributed 3 parameters to the 2-D velocity decoding model and, specifically, namely a row to the matrix H and a main diagonal element to the matrix R. Based on the BIC, the optimal subset size was determined to be 12 channels (circled as 430 in FIG. 4C), which remarkably matches the number of channels that contribute 90% of the total modulation depth (pointed to as 230 in FIG. 2B). The non-smooth and non-monotonic BIC curve reflects the fact that the relative improvement in decoding performance as a function of increasing the neural channel subset size is highly irregular when the subset size is small; increasing the subset from 1 to 2 channels does not reduce the estimation error as much as it increases the parameter space.

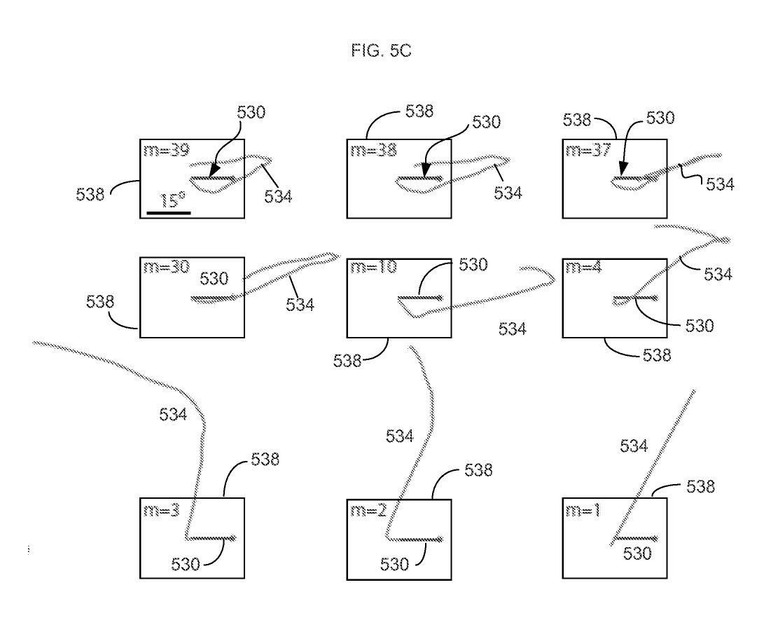

This situation when the most informative channels are “lost” are quite practical—it may occur due to relative micromovements of the recording array in relation to the subject and single-units. The empirical investigation of such phenomenon, as well as sensitivity of the decoder 150 to such loss demonstrated as expected that decoding performance deteriorates progressively as the highest modulation depth channels are removed from the decoder 150. The loss of the “best” channel, as can be seen from FIG. 5C, does not affect the results significantly. The results, illustrated in FIGS. 5A, 5B, show that although a small number (for example, m=5) of the highest modulated channels are sufficient to obtain accurate decoding (see FIG. 3A), the system is fairly resilient to the loss of highly modulated channels and suffers only a small loss in decoding performance, which loss could be easily compensated under feedback control.

The methodology of the estimation of modulation depth, discussed below, has substantially lower algorithmic complexity than conventionally used ranking based on decoding correlation, greedy search, or exhaustive search approaches, all of which involve multiple iterations of decoding analysis with the entire data set. As a practical measure of complexity, the time taken to perform a channel-subset selection using various schemes on the chosen computing platform (Desktop PC with 2×QuadCore Intel Xeon 3.2 GHz processor, 24 GB RAM, Windows 8.1 operating system, running Matlab software) was measured. The “exhaustive search” algorithm was not considered in this analysis to its prohibitive complexity, so “greedy search” was the most complex scheme in our analysis and served as the performance benchmark. As evidenced by Table I, the decoding-correlation-based selection had an order of magnitude lower complexity than greedy search, while modulation-depth-based channel selection of the present invention was 7 orders of magnitude lower. The complexity of the modulation depth scheme was within 2 orders of magnitude of that of random selection, which is the simplest available approach available. Therefore, the neural interface system configured to implement the modulation depth selection of neural channels offers the computational complexity that is vastly reduced in comparison with other schemes with only a small—if any—cost in performance, and thus offers a practically-superior channel selection mechanism.

Practical Confirmation of General Applicability.

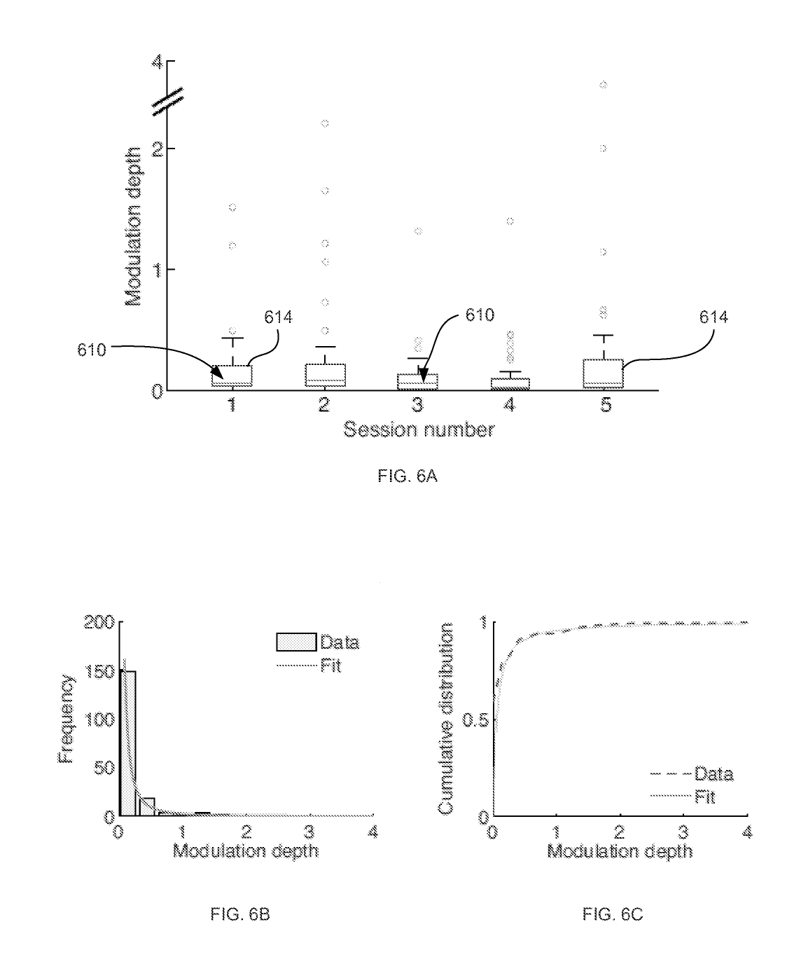

In order to verify that the embodiment of the invention is applicable across sessions (i.e., that the analysis of modulation depth characteristics was generalizable across sessions (each of which corresponded to a day of information acquisition from the same subject), the modulation depth distribution was analyzed in each of the five sessions. As illustrated in FIG. 6A, the observation of a long-tailed distribution was consistent across sessions. Here, line 610 denotes the median, while box 614 represents the interquatile range. A small number of outliers (i.e. channels with modulation depth several times higher than the median modulation depth) were observed in each session. The statistical properties of the modulation depth were characterized by estimating the best-fit distribution. For this analysis, the modulation depth data from all five sessions were pooled together and the probability distribution was estimated from the normalized histogram (FIG. 6B). Since the modulation depth distribution was highly asymmetric, the Doane's formula was used to compute the optimal number of histogram bins. A number of candidate probability distributions was investigated to obtain accurate statistical description of the data, and the maximum-likelihood parameter estimates for each candidate distribution were obtained; then the Anderson-Darling test was performed with the null hypothesis that the data were sampled from the estimated distribution. It was found that, of the distributions that passed the test with the null hypothesis at the 5% significance level, the generalized Pareto distribution provided the best fit (p=0.714), followed by the Weibull distribution (p=0.074). The alternative hypothesis was found to be true for the log normal, exponential, extreme value, Gaussian and several other distributions (p<10⁻⁶). Based on these results, it is concluded that the generalized Pareto distribution provides the most accurate statistical characterization of modulation depth with maximum likelihood estimates of shape parameter k=0.93 and scale parameter σ=0.06 [0.04,0.08] (mean and 95% confidence intervals). This is illustrated by the close agreement of the generalized Pareto probability density with the data (FIG. 6C). The Pareto distribution, originally formulated to describe unequal distribution of wealth among individuals, is widely used for modeling asymmetric, peaky data in many applications. In our analysis, the significant positive value of parameter k reflects heavy-tailed behavior, i.e. the presence of a small number of highly modulated channels.

The modulation depth statistics were also analyzed for each session individually and found the generalized Pareto distribution to provide the best fit to the data in each of the five sessions, followed by the Weibull distribution, further confirming earlier-discussed observations.

Embodiments of the present invention provide a biomedical system in which a signal subset selector unit extracts a subset of the available signal channels in correspondence with a threshold imposed on information content of the channels, and in which an operation of the decoder unit is governed by limiting the decoding operation to preprocessed signals received only from the channels in such subset. As a result of such selective decoding of the signal channels that the biomedical system employs from the overall number of available channels, the amount of data decoded by the system is drastically reduced, which is accompanied by the substantial reduction in dimensionality and complexity of the system as a whole. (For example, as seen in FIG. 4C, with the choice of the 12 best or optimal channels from the set of 39 channels the reduction coefficient is at least 27/39; the reduction coefficient grows as a function of the initial number of all available channels in the system: with the choice of 12 best channels out of the initial 200 channels, for example, the reduction coefficient is at least 188/200). The SNR of the multivariate neural interface system of the invention is dimensionless and is consistent across sensory motor tasks, behavioral state variables, neural signals, and sample rates, due to which it can be used for comparative analysis of a variety of system configurations and experimental conditions. The use of embodiments of the invention demonstrates that the metric of the modulation depth, used as a metric to rank the information content of the channels and select the optimal subset in a neural interface system produces a system, can be used to analyze and compare the information content and decoding potential of multiple neural signal modalities, such as binned spike-rates and LFP, and also signals recorded from different areas in the brain. The total modulation depth summed across the ensemble could be used in conjunction with a pre-specified threshold to determine whether the information quality is poor and filter recalibration should be initiated.

In one embodiment, most of the computational procedures required to estimate modulation depth are performed as part of the standard Kalman filter calibration process and therefore incur little additional computational cost. The only additional computation required to obtain the SNR matrix, S, includes relatively simple matrix operations, such as matrix multiplications involving the measurement noise covariance matrix that usually has a diagonal structure. Compared to alternative approaches for computing channel modulation measures and ranks (such as those involving neural decoding with correlation-based greedy subset selection), the embodiments of the invention have substantially lower computational complexity. (As evidenced by the experimental data from Table I, for example, the time required for the otherwise equal data-processor to perform the required computations was reduced by about six orders of magnitude for the embodiment of the invention as compared with the conventional greedy search, and, as was already alluded to above in a specific example of choosing the 12 best channels out of the set of 39 channels, the complexity of the system was reduced by at least 27/39. Accordingly, not only the embodiment of the invention ties the mathematical operation(s) to the processor's ability to process digital and/or analog data, but it improves the functioning of the processor itself by causing it to use less storage memory than required for performing the same tasks by the systems of related art, results in faster computation time (i.e., uses less computing power) without sacrificing the quality of the resulting operation of an endeffector (such as, in a non-limiting ex ample, a prosthetic device), and produces a simplified neural interface system.) It is therefore highly suited to real-time neural decoding and high-throughput offline analyses with high-dimensional state or observation vectors or large number of temporal samples. (For the purposes of this disclosure and accompanying claims, a real-time performance of a system is understood as performance which is subject to operational deadlines from a given event to a system's response to that event.) Indeed, in stark contradistinction with the commonly used greedy search algorithm, which requires hours for optimal channel identification based on pre-recorded data, the embodiment of the present invention perform the required assessment during the actual operation of the biomedical system, thereby enabling the selector 140 and the decoder 150 to govern the endeffector in a matter of seconds at most.

The low computational cost makes repeated estimation of modulation depth possible as may be required during online real-time decoding for performance monitoring or closed-loop recalibration. In a nonstationary setting, the time-varying form of the system SNR and modulation depth can provide an instantaneous measure of the signal quality and the importance of a particular channel. The nonstationary setting may be defined by a situation when the statistics of neural signals is changing in time, and/or when the mapping of neural signals to behavioral variables—such as the velocity of cursor in the experiments discussed above—is changing in time (additional insight is provided by the discussion of Eq. (12) in the Methodology section, below).

### Methodology.

Using the two-dimensional motor imagery task of a prosthetic device (such as a prosthetic arm) as an example, the intended movement kinematics is estimated from the neural activity during the performance of such two-dimensional task with the used of a state-space model. The vector time-series of observations (for example, single-unit binned spike-rates) at time t are denoted with m-dimensional vector y(t), where m is the number of channels (for example, single-units). The intended movement velocity is represented by the n-dimensional latent variable, x(t), for example with the use of a velocity encoding model for primary motor cortical neurons (D. W. Moran et al., J. Neurophysiol., vo. 82, no. 5, pp. 2676-2792, November 1999). This latent variable is to be estimated from spike-rates y(t) using a state-space paradigm. The intended velocity may be considered in the two-dimensional Cartesian coordinate system (n=2 in this case), while m represents the neuronal ensemble size. The modulation depth of each of the m neural channels is determined as described below.

Dynamical System Model

The proposed methodology considers a continuous-time linear time-invariant (LTI) Gaussian dynamical system model consisting of a latent multivariate random process, x(t), with Markovian dynamics, related to multivariate observations, y(t). Such state-space model is expressed as:

x(t)=Fx(t)+ω(t) (1)

y(t)=Hx(t)+v(t) (2)

where tεR⁺, x(t) is the n-dimensional state vector, y(t) is the m-dimensional observation vector, ω(t) is the n-dimensional process noise vector, v(t) is the m-dimensional measurement noise vector, and F(t) and H(t) are the n×n system matrix and nxm observation matrix, respectively. The noise processes have mean zero, i.e., E{ω(t)}=E{v(t)}=0. The noise covariances are E{ω(t)ω′(τ)}=Q_{c}δ(t−τ), E{v(t)v′(τ)}=R_{c}δ(t−τ), and E{ω(t)v′(τ)}=0. Here, δ(•) is the Dirac delta function and ( )′ denotes matrix transpose. Q_{c} is assumed to be a symmetric positive semidefinite matrix and R_{c} is assumed to be a symmetric positive definite matrix. The observations are also assumed to have zero-mean, i.e., E {y(t)}=0.

Modulation Depth

In Eq. (2), Hx(t) is defined as the signal and v(t) is defined as the noise. The modulation depth (MD) of a channel is further defined as the signal-to-noise ratio (SNR) of the channel, that is the ratio of the signal and noise covariances. For the purposes of the multivariate state-space model, the mxm signal and noise covariance matrices are denoted as Λₛ(t) and Λₙ(t), respectively. Then, the m×m time-varying SNR matrix of observations y(t) can be expressed as:

$$\begin{matrix}S ( t ) = {Λ}_{S} ( t ) {Λ}_{n}^{- 1} ( t ) = E \{ {[ Hx ( t ) ] [ Hx ( t ) ]}^{′} \} = {[ E \{ v ( t ) {v ( t )}^{′} \} ]}^{- 1} = \\ = {HP}_{C} ( t ) {H}^{′} {R}_{C}^{- 1}\end{matrix} \qquad ( 3 )$$

where the n×n matrix P_{c}(t) is defined by x(t)˜N(μ_{c}(t),P_{c}(t)).

The Eq.(3) defines the SNR of a continuous-time multiple-input multiple-output state-space model. The (i,j)th and (j,i)th elements of the symmetric matrix Λₛ(t) represent the signal covariance between ith and jth channels at time t. Similarly, the (i,j)th and (j,i)th elements of Λₙ(t) represent the noise covariance between channels i and j. If channels i and j have uncorrelated noise, then R is a diagonal matrix. Then the (i,j)th element of S(t) represents the signal covariance of channels i and j relative to the noise covariance of channel j, whereas the (j,i)th element represents the signal covariance of channels i and j relative to the noise covariance of channel i. It is appreciated that regardless of the structures of Λₛ(t) and Λₙ(t), the ith diagonal element of S(t) represents the SNR of the ith channel at time t.

For the homogeneous LTI differential equation [symbol] (t)=Fx(t), the fundamental solution matrix is Φ(t) such that [symbol] (t)=4:1)(t)x(0), where the state-transition matrix Φ(t,τ)=Φ(t)Φ⁻¹(τ) represents the system's transition to the state at time t from the state at time τ and Φ(t,0)=Φ(t). For the dynamical system in Eq. (1), the state transition matrix Φ(t,τ)=e^{F(t-τ)} depends only on the time difference (t−τ). The solution to the nonhomogeneous differential Eq. (1) is

$$\begin{matrix}x ( t ) = Φ ( t , {t}_{0} ) x ( {t}_{0} ) + {\int }_{{t}_{0}}^{t} Φ ( t , τ ) ω ( τ ) d τ & ( 4 ) \\ = exp \{ F ( t - {t}_{0} ) \} x ( {t}_{0} ) + {\int }_{{t}_{0}}^{t} exp \{ F ( t - τ ) \} ω ( τ ) d τ & ( 5 )\end{matrix}$$

The covariance matrix of x(t) in Eq. (4) is given by

P_{C}(t)=Φ(t,t₀)P_{C}(t₀)Φ′(t,t₀)+∫ₜ_{₀}^{t}ψ(t,τ)Q_{C}Φ′(t,τ)dτ (6)

Model Discretization.

The system model in Eq. (5) can be discretized by sampling at tₖ=kΔt=tₖ₋₁+Δt for k=1, . . . , K, so that

$$x ( {t}_{k} ) = {e}^{F Δ t} x ( {t}_{k - 1} ) + {\int }_{{t}_{k - 1}}^{{t}_{k}} {e}^{F ( {t}_{k} - τ )} ω ( τ ) d τ . \qquad ( 7 )$$

The state-transition matrix Φ=e^{FΔt} is defined, so the equivalent discrete-time system can be represented by

x[k+1]=Φx[k]+w[k] (8)

y[k]=Hx[k]+v[k] (9)

where kε [symbol] ⁺, E{w[k]}=E{v[k]}=0, E{w[k]w′[l]}=Q_{d}δ[k,l], E{v[k]v′[l]}=R_{d}δ[k,l], and δ[•,•] is the Kronecker delta function. The state x[k] is a Gaussian random variable initialized with x[0]˜N(μ_{d}[0],P_{d}[0]).

From the power series expansion for a matrix exponential,

Φ=e^{FΔt}=I+FΔt+O(Δt²)≈I+FΔt. (10).

According to (J. M. Mendel, in Lessons in Estimation Theory for Signal Processing, Communications, and Control, 2d Ed., Prentice-Hall, 1995), the covariance of x[k+1] in Eq.(8) can be written as

P_{d}[k+1]=ΦP_{d}[k]Φ′+Q_{d}. (11)

The equivalent discrete-time SNR is determined by discretizing Eq. (3) and substituting the discrete-time covariances, so that

$$S [ k ] = \frac{1}{Δ t} {HP}_{d} [ k ] {H}^{′} {R}_{d}^{- 1} . \qquad ( 12 )$$

Notably, for the above continuous-time and discrete-time system models to be equivalent, the approximations Q_{d}≈Q_{c}Δt and R_{d}≈R_{c}/Δt have to be used, neglecting O(Δt²) terms. When these conditions are observed, the continuous- and discrete-time covariance matrices are equal, i.e. P_{c}(t)=P_{d}[k].

In the following, certain classes of linear dynamical systems are considered that occur frequently in practice and that admit special forms for modulation depth estimation, such as a steady-state system and a time-variant system.

Steady-State Systems.

On differentiating Eq. (6) and using the relation [symbol] Φ(t,t₀)=FΦ(t,t_{( )}), the solution produces

[symbol]

_{C}(t)=FP_{C}(t)+P_{C}(t)F′+Q_{C}. (13)

For a stable LTI continuous-time system at steady-state, the Eq. (13) can be re-written as lim_{t→∞} P_{C}(t)=0. Thus, the steady-state covariance, P̅_{C}=lim_{t→∞}P_{C}(t), is given by the continuous-time algebraic Lyapunov equation

FP̅_{C}+P̅_{C}F′+Q_{C}=0 (14)

If λᵢ(F)+λⱼ(F)≠0 ∀i,jε{1, . . . , n}, where λᵢ(F) denotes an eigenvalue of F, the solution P̅_{C} to the above equation is given by

$${C}_{} = {\int }_{0}^{∞} {e}^{F τ} {Q}_{C} {e}^{{F}^{′} τ} d τ . \qquad ( 15 )$$

Similarly, let us consider the discrete-time covariance. The system is stable if |λᵢ(Φ)|<1∀iε{1, . . . , n}, where λᵢ(Φ) denotes an eigenvalue of Φ. For a stable LTI system, the steady-state covariance,

$${lim}_{k -> ∞} {P}_{d} [ k ] = {d}_{} ,$$

can be obtained from Laubach et al. (Nature, vol. 405, no. 6786, pp. 567-571, June 2000) and expressed in terms of the discrete-time algebraic Lyapunov equation or Stein equation

ΦP̅_{d}Φ′−P̅_{d}+Q_{d}=0 (16)

If λᵢ(Φ)λᵢ(Φ)≠1∀i,jε{1, . . . , n}, the solution to the above equation is given by

$${d}_{} = {\sum }_{k = 0}^{∞} {Φ}^{k} {{Q}_{d} ( {Φ}^{′} )}^{k} . \qquad ( 17 )$$

Eqs. (15) and (17) can be solved numerically using, for example, the Schur method (A. J. Laub, IEEE Automatic Control, vol. 24, no. 6, pp. 913-921, December 1979). The steady-state SNR,

$$= {lim}_{k -> ∞} S [ k ] ,$$

can therefore be expressed in terms of discrete-time system parameters as

$$= \frac{1}{Δ t} {HP}_{d} {H}^{′} {R}_{d}^{- 1} . \qquad ( 18 )$$

Time-Variant Systems

The expression in Eq. (12) allows to assess the instantaneous SNR of the neural signal observations at the k^{th} time-step (i.e., time equal to k). This expression can also be used to estimate the SNR of a time-varying or non-stationary system. Expressing the state transition matrix as Φ[k+1,k], the observation matrix as H[k], and the process noise covariance matrix as Q_{d}[k], the propagation of the time-varying state covariance of a state-space system can be written as

P_{d}[k+1]=Φ[k+1,k]P_{d}[k]ψ′[k+1,k]d+Q_{d}[k] (19)

By recursive expansion of Eq. (19) and using the property

$$Φ [ k ] = Φ [ k , 0 ] = {Π}_{i = k}^{1} Φ [ i , i - 1 ]$$

one can write

P_{d}[k]=Φ[k]P_{d}[0]Φ′[k]+Σᵢ₌₀^{k-1}ψ[k,k−i]Q_{d}[k−i−1]Φ′[k,k−i] (20)

If the process noise is wide-sense stationary, then Q_{d}[k]=Q_{d}, and the above equation simplifies to

P_{d}[k]=Φ[k]P_{d}[0]Φ′[k]+Σᵢ₋ᵢ₌₀^{k-1}Φ[k,k−i]Q_{d}Φ′[k,k−i] (21)

The above expressions provide an estimate of the state covariance at time k in terms of the initial state covariance and the process noise covariance.

Estimation of the Trajectory of SNR.

Estimation So far we have considered SNR estimation for two classes of linear dynamical systems: the instantaneous SNR S[k] for a time-varying system, and the steady-state SNR⁻S for a time-invariant and stable system. These quantities provide measures of instantaneous information flow between the two multivariate stochastic processes under consideration, namely x[k] and y[k]. Following the nomenclature used in recent work on mutual information estimation for point process data (see S. A. Pasha and V. Solo. In Proc. IEEE Eng. Med. Biol. Conf., 2012, pp. 4603-4606), this measure is referred to as the marginal SNR. Now is considered the alternate problem of information flow between the entire trajectories of the random processes (as opposed to the instantaneous or marginal measure). To rephrase, the trajectory SNR is a measure of the SNR conditioned on the entire set of observations for k=1, . . . , K. This formulation is useful for SNR estimation by offline, batch processing of data for a time-varying system.

Given the time-varying observation matrix H[k] and state-transition matrix Φ[k], and assuming the noise processes w[k] and v[k] are zero-mean and stationary with covariance matrices Q_{d} and R_{d} respectively, we can obtain the m×m trajectory SNR matrix as

[symbol]

= [symbol] [symbol] _{d} [symbol] ′R_{d}⁻¹ (22)

where [symbol] =[H[1], . . . , H[k]] is an m×nK block matrix and

$${𝒫}_{d} = [ \begin{matrix}{P}_{d} [ 1 , 1 ] & … & {P}_{d} [ 1 , K ] \\ ⋮ & ⋱ & ⋮ \\ {P}_{d} [ K , 1 ] & … & {P}_{d} [ K , K ]\end{matrix} ] \qquad ( 23 )$$

is a nK×nK block matrix. Here P_{d}[i,j] denotes the covariance between x[i] and x[j] for i,jε{1, . . . , K}, i≠j, defined as

$${P}_{d} [ i , j ] = \{ \begin{matrix}{P}_{d} [ i ] {Φ}^{′} [ j ; i ] for i < j \\ {P}_{d} [ i ] for i = j \\ Φ [ i ; j ] {P}_{d} [ j ] for i > j\end{matrix} . \qquad ( 24 )$$

Colored Measurement Noise.

Now discussed is the case in which the multivariate measurement noise v[k] in the state-space model of the present invention is a colored noise process (i.e. the one correlated with itself over time). Assume that v[k] is zero-mean and stationary. According to Wold's decomposition theorem, v[k] can be represented as an m-dimensional vector autoregressive process of order p, i.e. VAR(p), given by

$$v [ k ] = {\sum }_{i = 1}^{p} {ψ}_{i} v [ k - i ] + ζ [ k ] , \qquad ( 25 )$$

where ζ[k] is an m-dimensional zero-mean white noise process with covariance E{ζ[i]ζ[j]}=Wδ[i,j], and ψᵢ is an m×m VAR coefficient matrix. The above VAR(p) process can be expressed in VAR(1) companion form as

V[k]=ψV[k−1]+Z[k] (26)

where V[k] is the mp×1 vector

V[k]=[k],[v′[k],v′[k−1], . . . ,v′[k−p+1]]′ (27)

Z[k] is the mp×1 whitened noise vector

Z[k]=[ζ′[k],0, . . . ,0]′ (28)

with covariance E{Z[i]Z′ [j]}=diag{Wδ[i,j],0}, Ψ is the mp×mp VAR(p) coefficient matrix

$$Ψ = [ \begin{matrix}{ψ}_{1} & {ψ}_{2} & … & {ψ}_{p - 1} & {ψ}_{p} \\ {I}_{m} & 0 & … & 0 & 0 \\ 0 & {I}_{m} & … & 0 & 0 \\ ⋮ & ⋮ & ⋱ & ⋮ & ⋮ \\ 0 & 0 & … & {I}_{m} & 0\end{matrix} ] , \qquad ( 29 )$$

and Iₘ denotes the mxm identity matrix.

With this formulation, one can augment the state-space with [k] so that the augmented state vector has dimensions (n+m)×1 and is given by X[k]=[x′[k]], V′[k]. The SNR estimation methodology described earlier can then be applied directly. It should be noted that when using this state augmentation approach for noise whitening, the parameter space can increase substantially if the measurement noise is modeled as a high-order VAR process, which may result in over-fitting. The optimal VAR model order, p, can be estimated using standard statistical methods based on the Akaike Information Criterion or Bayesian Information Criterion.

Uncorrelated State Vector.

We now consider the special case that the n states, represented by the random n-dimensional vector x[k] at discrete time k, are mutually uncorrelated. Practically, this situation may occur in neural interface systems when, for example, the decoder's state vector has three components representing intended movement velocity in three-dimensional Cartesian space. Then one has

Φ=diag{Φ_{1,1}, . . . , φ_{n,n}} and Q_{d}=diag{q_{1,1}, . . . , q_{n,n}}. The steady-state covariance of the state, given by Eq. (17), simplifies to

P̅_{d}=Q_{d}(I−ΦΦ′)⁻¹ (30)

where we have used the formula for the Neumann series expansion for a convergent geometric series, and the fact that λᵢ(ΦΦ′)=λᵢ(Φ)λᵢ(Φ), and therefore |λᵢ(ΦΦ′)|<1, so that ΦΦ′ to is stable. Eq. (30) can be re-written as

$${\overline{P}}_{d} = diag \{ \frac{{q}_{1 , 1}}{1 - {φ}_{1 , 1}^{2}} , … , \frac{{q}_{n , n}}{1 - {φ}_{n , n}^{2}} \} \qquad ( 31 )$$

The diagonal elements in P̅_{d} then correspond to the covariances of a set of n mutually independent first-order autoregressive processes each with autoregression coefficient φ_{i,i} and white noise variance q_{i,i} for i=1, . . . , n. For this asymptotically stable system with a diagonal state-transition matrix, the steady-state SNR in Eq. 918) can be obtained as

$$= \frac{1}{Δ t} {{HQ}_{d} ( I - {ΦΦ}^{′} )}^{- 1} {H}^{′} {R}_{d}^{- 1} . \qquad ( 32 )$$

When R_{d} is also diagonal, the SNR of the ith channel is given by the ith diagonal element of ⁻S for i=1, . . . , m, i.e.

$${s}_{i} = {s}_{i , i} = \frac{1}{Δ t {r}_{i , i}} {\sum }_{j = 1}^{n} \frac{{q }_{j , j}}{( 1 - {ϕ}_{j , j}^{2} )} {h}_{i , j}^{2} . \qquad ( 33 ) .$$

Note that if the state vector includes, for example, position, velocity and acceleration in Cartesian space, the condition of independence is violated due to coupling between the states, which is reflected in the non-zero off-diagonal entries of Φ.

The SNR expressions of Eqs. (12) and (32) include the sampling interval, At, since the quantities on the right-hand side correspond to the discrete-time system representation. We now show that if the SNR estimate is converted to the original continuous-time representation, At factors out of the SNR equation. To demonstrate this, the discrete-time variables in Eq. (32) can be transformed into the corresponding continuous-time variables by substituting Φ=I+FΔt, Q_{d}=Q_{c}Δt, and R_{d}=R_{c}/Δt, and neglecting higher order terms O(Δt²), so that

[symbol]

=−HQ_{C}[F+F′]⁻¹H′R_{C}⁻¹. (34)

The variables in the above expression correspond to the continuous-time system model.

The above expression shows that the SNR is consistent when derived using equivalence relations for continuous- and discrete-time systems.

It is understood, therefore, that the idea of the present invention has been implemented to define a channel content of a neural interface system based on estimation of the modulation depth (of observation signals acquired by the system through such channels) with the use of a multivariate state-space model. This modulation depth estimator is closely related to the Kalman filtering paradigm and is suitable for channel ranking in a multichannel neural system. In contrast with greedy selection and information theoretic measures used by the related art, the proposed methodology state-space modulation depth estimation scheme has high computationally efficiency and thus provides a method for real-time variable subset selection useful for closed-loop decoder operation. Due to these characteristics, the use of the modulation depth estimation and channel-ranking scheme according to an embodiment of the invention is highly suitable for both offline neuroscience analyses and online real-time decoding in neural interfaces. This approach enables optimal signal subset selection and real-time performance monitoring in future neural interfaces with large signal spaces.

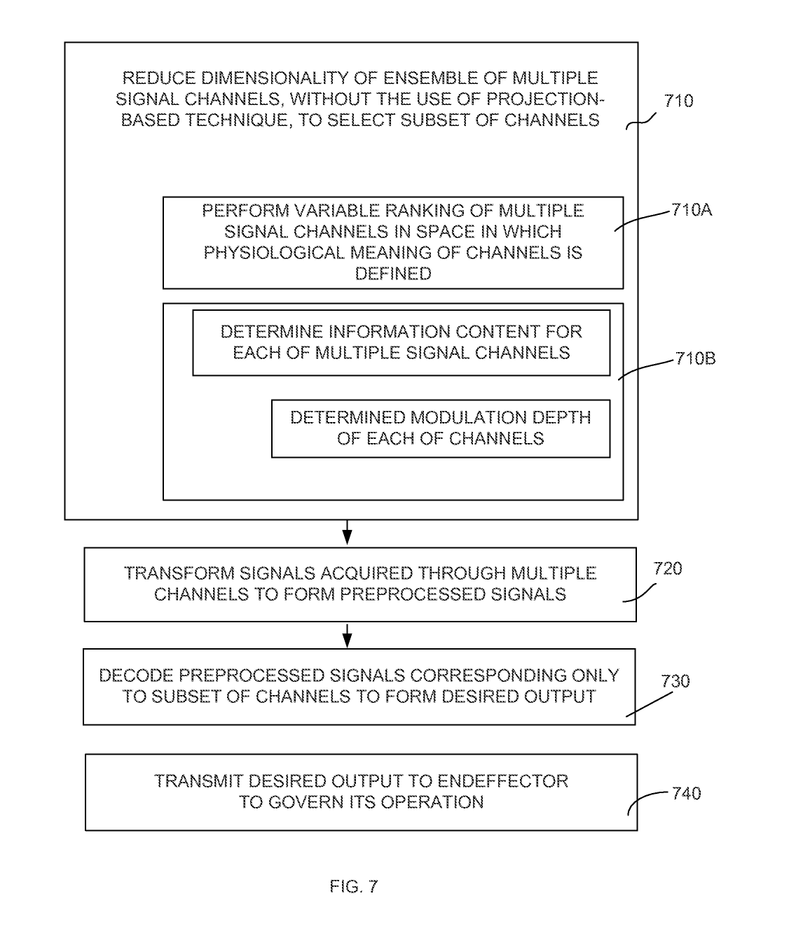

FIG. 7 presents a schematic flow-chart of an embodiment of a method of the invention, which includes step 710, at which a dimensionality of the ensemble of multiple signal channels of the biomedical system is reduced without the use of a projection-based technique. As a results of such reduction, a subset of the overall number of channels is defined. The reduction of dimensionality is carried out by ranking the channels, as step 710A, in the original space in which their physiological meaning is defined, and by determining information content of each of the channels at step 710B (the latter can include, in one implementation, the determination of modulation depth of the channels). The signals acquired through all the channels are preprocessed according to the defined criteria, at step 730, and the preprocessed signals carried only by the channels selected to form the subset of channels is further decoded, at step 740, to generate a desired output. This output is further transferred or transmitted to an endeffector at step 750 to cause the endeffector to perform its function.

Overall, this disclosure discussed a generalized modulation depth measure using the state-space framework that quantifies the tuning of a neural signal channel to relevant behavioral covariates. For a dynamical system, computationally efficient procedures for estimating modulation depth from multivariate data were developed. It was shown that the chosen metric can be used to rank neural signals and select an optimal channel subset for inclusion in the neural decoding algorithm. A scheme for choosing the optimal subset based on model order selection criteria was applied to neuronal ensemble spike-rate decoding in neural interfaces, using our framework to relate motor cortical activity with intended movement kinematics. With offline analysis of intracortical motor imagery data obtained from individuals with tetraplegia using the BrainGate neural interface, it was demonstrate that the proposed variable selection scheme is useful for identifying and ranking the most information-rich neural signals. The proposed approach offers several orders of magnitude lower complexity but virtually identical decoding performance compared to greedy search and other selection schemes. The discussed statistical analysis showed that the modulation depth of human motor cortical single-unit signals is well characterized by the generalized Pareto distribution. The proposed channel-selection scheme has wide applicability in problems involving multisensor signal modeling and estimation in biomedical engineering systems.

It will be readily apparent to those skilled in this art that various changes and modifications of an obvious nature may be made, and all such changes and modifications are considered to fall within the scope of the present invention.

For example, the determination of the modulation depth may be useful in a wide range of systems neuroscience applications beyond movement-related neural interfaces. It is widely applicable to analyses of neural systems involving a set of behavioral correlates and multichannel recordings.

In a related embodiment, for example, the discussed methodology can be applied directly to the measurement of sensory response of a primary visual cortex neuron to a visual stimulus. Here, the method of the invention is used to neuroscience analysis of the neural encoding of a given sensory input. For example, a simple, known sensory stimulus, such as a visual input or an image (for example, in the simplest case, a contrast grating such as a series of thick black and white lines) which is varied in a known way (e.g. the orientation of the grating changes over time). The animal is made to observe this known image, and the brain activity (in a visual area of the brain, such as the primary visual cortex) generated in response to this image is recorded. The recording is effectuated with a set of channels (multiple electrodes on a microelectrode array implanted into the cortex), each with an electrical voltage time-series containing spikes from individual neurons, or as a set of two-photon image pixels, as described above. As a result, multi-channel data are generated in response to the stimulus, from which the channels that are the most responsive to the applied stimulus (i.e. have the highest stimulus-related information or greatest modulation depth) are then identified. The method as described above can be used for this purpose directly (that is, without any modification).

The example of two-photon imaging to micron-resolution functional imaging is described in detail in U.S. Pat. No. 8,903,192 which is incorporated by reference herein. Briefly, a part of cortical tissue is labeled with a fluorescent protein that indicates calcium activity, and a two-photon microscope is used to measure the fluorescence. Calcium dynamics are a measure of neural activity in the brain. This imaging methodology is now being used a lot for micron-resolution functional imaging (i.e. observing response to some input, as opposed to just the static structure) of brain tissue.

In the above setting, the measure of the modulation depth of the acquired signals is used to quantify the response evoked in that neuron by the applied stimulus, and results in a tuning curve estimate for that neuron. Repeating such analysis across a neuronal ensemble can help in comparative analysis of neuronal tuning properties. Alternatively or in addition, the proposed methodology can be applied to any neuroscience experiment in which the underlying system can be expressed in the form of a state-space model that involves any of the intracortical (spike-rate, multiunit threshold crossing rate, analog multiunit activity, or local field potential), epicortical (elecrtrocorticogram), or epicranial (electroencephalogram or functional magnetic resonance imaging) neural signals.

In a related embodiment, the proposed methodology can be modified to incorporate a temporal lead or lag between the latent state and the neural activity. As the related art demonstrated (L. Paninski et al., J. Neurophysiol., vol. 91, no. 1, pp. 515-532, January 2004), motor cortical single-unit spike signals typically lead movement by about 100 ms in able-bodied monkeys and are more variable in humans with paralysis (W. Truccolo et al., J. Neurosci., vol. 28, no. 5, pp. 1163-1178, January 2008), and similarly LFP beta rhythm leads movement due to its encoding of movement onset information (J. P. Donoghue, Neuron, vil. 60, no. 3, pp. 511-521, November 2008). The analysis of all such temporal effects can be carried out through the covariance structure of model residuals or the likelihood function, and the optimal lags can be included in the observation equation on a per-channel basis, after which modulation depth can be estimated as disclosed in this application.

In another related embodiment, including the fMRI data analysis, the above-described method is used for this problem of pixel selection in an fMRI image. Specifically, a linear regression model relating the input and output (similar to Eqs. 2 or 9 above) can be defined for each pixel (or signal channel). The identification of which pixel(s)/channel(s) contain meaningful information (i.e. are responsive to a stimulus) and should be analyzed is now performed based on the values and significance of the regression coefficients (for example, by using standard statistical hypothesis testing procedures). In a specific case, the same method is employed for identifying neurons from a two-photon calcium image of the brain, for example. Furthermore, in contrast with regression-based methods which require the system (for example, a brain) to be time-invariant, the method of the present invention is rooted in a dynamical system approach (of which the static or time-invariant system is a subclass) and, therefore, the selection of fMRI pixel(s)/channel(s) with the use of an embodiment of the invention is successful and/or operational even under time-varying situations.

In another related embodiment, the information content of each of the available channels is being determined as discussed above, and the channels are ranked based on the determined information content. Then, the first X channels (having the highest levels of information content) are chosen or determined according to a criterion that is not connected with the determination or presence of the information content of the signal channels according to the idea of the invention. Such situation may arise, for example, in a specific case when it is known a priori that only X signal channels can be kept because of the limitations of the electronic circuitry of the system of the invention (such as, for example, physical width of a hardware data-bus)

Embodiments of the biomedical system of the invention have been described as including a processor controlled by instructions stored in a memory. The memory may be random access memory (RAM), read-only memory (ROM), flash memory or any other memory, or combination thereof, suitable for storing control software or other instructions and data. Some of the functions performed by the discussed embodiments have been described with reference to flowcharts and/or block diagrams. Those skilled in the art should readily appreciate that functions, operations, decisions, etc. of all or a portion of each block, or a combination of blocks, of the flowcharts or block diagrams may be implemented as computer program instructions, software, hardware, firmware or combinations thereof. Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

In addition, it is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

1. A biomedical system for transforming activity signals acquired from a biological tissue, the system comprising:
an input interface unit simultaneously receiving a multiplicity of raw signals from the array of signal channels;
a preprocessing electronic circuitry configured to transform said raw signals to refined signals by at least amplifying said raw signals, filtering said raw signals to reduce noise contained therein, and detecting a predetermined feature in said raw signals;
a estimator unit configured to determine information content of each of the signal channels from said array; and
a channel subset selector in operable communication with the estimator unit to receive values representing said information content of the signal channels and, based on said valued that have been ordered, generate a marker representing a limit on a number of said signal channels to be used in the system.

2. A biomedical system according to claim 1, wherein said activity signals include neural activity signals and wherein said raw signals include electrical signals.

3. A biomedical system according to claim 1, wherein said information content includes a modulation depth of each of the signal channels.

4. A biomedical system according to claim 3, wherein said channel subset selector, in operation, receives values of modulation depths of each of said signal channels from the array and generates said marker when a cumulative sum of these values, that have been ordered in a descending order, exceeds a predefined threshold.

5. A biomedical system according to claim 1, wherein said predetermined feature includes a spike in a raw signal.

6. A biomedical system according to claim 1,
wherein the signal subset selector is configured to
form a first sequence containing said values in a descending order,
form a second sequence containing numbers of channels respectively corresponding to values in the first sequence, and
identify numbers of channels from the first sequence according to a criterion representing a physical factor unrelated to the determination of the information content of the signal channels,
and
wherein said marker includes a number of channels identified by the signal subset selector from the array of signal channels.

7. A biomedical system according to claim 1, wherein the estimator unit contains electronic circuitry programmed to determine a modulation depth, of a chosen signal channel from the array, as a ratio of i) a response of said chosen signal channel to a behavioral variable to ii) a spontaneous activity of said chosen signal channel and noise.

8. A biomedical system according to claim 6, wherein said behavioural variable include a pre-determined input provided to the biological tissue.

9. A biomedical interface system according to claim 7, wherein said pre-determined input includes at least one of an optical stimulus, an auditory stimulus, a tactile stimulus, a gustatory stimulus, and an olfactory stimulus.

10. A biomedical system according to claim 1, further comprising
a decoder unit, in electrical communication with the channel subset selector and the preprocessing electronic circuitry, and
an endeffector operably connected to the decoder unit,
said decoder unit being configured to acquire first refined signals corresponding only to signal channels from a subset of the array identified by the marker, and to decode said first refined signals in a fashion that is suitable to generate a required response from the endeffector subjected to decoded refined signals.

11. A biomedical system according to claim 10, wherein said endeffector includes a tissue.

12. A biomedical interface system according to claim 10, wherein the decoder unit decodes said refined signals to generate such a physiological response from said tissue which mimics a pre-determined input that has been provided to the biological tissue, and based on which the information content of a channel from the array is determined by the estimator unit.

13. A biomedical system according to claim 10, wherein, in operation, a duration of time between first time and second time is real time, the first time corresponding to a moment of receiving the multiplicity of raw signals by the input interface unit, the second time corresponding to a moment of decoding of all of said first refined signals.

14. A method for operating a biomedical system, the method comprising:
reducing a dimensionality of an ensemble of multiple signal channels, through which activity signals are acquired from a biological tissue that has been subjected to an input, by selecting a signal channel subset, from said ensemble, without employing a projection-based technique; and
decoding a subset of said activity signals acquired through said signal channel subset to obtain a first output that differs in a predetermined fashion from to a second output, the second output being a result of decoding of activity signals acquired through the entire ensemble of multiple signal channels.

15. A method according to claim 14, wherein the first output includes at least one of an optical signal, an electrical signal, a magnetic signal, a mechanical signal, and a audio signal.

16. A method according to claim 14, wherein said first output includes a decoding correlation obtained as a result of decoding said subset of said activity signals, wherein said second output includes a decoding correlation obtained as a result of decoding all activity signals.

17. A method according to claim 14, further comprising determining an optimal number of channels in said signal channel subset based on a Bayesian information criterion.

18. A method according to claim 14, wherein said reducing dimensionality includes performing variable ranking of the multiple signal channels in an original signal space, in which a physiological meaning of said multiple signal channels is defined, and is devoid of transforming said multiple signal channels into a space that does not represent the physiological meaning.

19. A method according to claim 14, wherein said reducing dimensionality include determining information content for each of said multiple signal channels.

20. A method according to claim 19, further comprising
forming a first sequence containing values of modulation depth for all of the multiple signal channels from the ensemble in a descending order,
forming a second sequence containing numbers of said multiple signal channels respectively corresponding to said values in the first sequence, and
identifying numbers of said multiple signal channels from the first sequence that correspond to those values, from the first sequence, a first sum of which exceeds the predefined threshold, the first sum obtained by adding successive values of modulation depth from the first sequence.

21. A method according to claim 14, wherein said reducing dimensionality includes determining modulation depths of the multiple signal channels and ranking said multiple signal channels based on determined modulation depths.

22. A method according to claim 21, wherein said determining modulation depths includes determining a modulation depth of a chosen signal channel as a ratio of i) a response of said chosen signal channel to a pre-determined input provided to the behavioral variable, to ii) a spontaneous activity of said chosen signal channel and noise.

23. A method according to claim 22, further comprising
transforming raw signals acquired through the ensemble to refined signals by at least amplifying said raw signals, filtering said raw signals to reduce noise contained therein, and detecting a predetermined feature in said raw signals; and
decoding only those refined signals that correspond to multiple signal channels identified by said numbers, said decoding effectuated in a fashion that is suitable to generate a required response from an endeffector subjected to decoded refined signals.

24. A method according to claim 23, wherein said decoding includes decoding effectuated in a fashion suitable to generate a required response from the endeffector that includes a tissue, and further comprising subjecting the biological tissue to a pre-determined input that includes at least one of an optical stimulus, an auditory stimulus, a tactile stimulus, a gustatory stimulus, and an olfactory stimulus.

25. A method according to claim 24, wherein said decoding includes decoding effectuated to generate a physiological response from said tissue, which physiological response mimics the pre-determined input to the biological tissue.

26. A method according to claim 14,
wherein the signal subset selector is configured to
form a first sequence containing said values in a descending order,
form a second sequence containing numbers of channels respectively corresponding to values in the first sequence, and
identify numbers of channels from the first sequence according to a criterion representing a physical factor unrelated to the determination of the information content of the signal channels,
and
wherein said marker includes a number of channels identified by the signal subset selector from the array of signal channels

27. A method according to claim 14,
wherein the reducing a dimensionality of an ensemble of multiple signal channels includes reducing a number of pixels of an image of the biological tissue based on information content of each of said pixels, and
wherein the decoding a subset of the activity signals acquired through the signal channel subset includes decoding information contained in a subset of the pixels of the image.
